(19) United States
(12) Patent Application Publication
(10) Pub. No.: US 2012/0053330 A1
(43) Pub. Date: Mar. 1, 2012

(54) Chemical Process

(76) Inventors: Daniel Edward Patterson, Research Triangle Park, NC (US); Michael S. McClure, Research Triangle Park, NC (US); Jeremiah David Powers, Research Triangle Park, NC (US); Claire Frances Crawford, Hertfordshire (GB); Malcolm Brian Berry, Hertfordshire (GB)

(21) Appl. No.: 13/265,001

(22) PCT Filed: Apr. 29, 2010
(86) PCT No.: PCT/US10/32893

§ 371 Date: Oct. 18, 2011

Related U.S. Application Data
(60) Provisional application No. 61/174,097, filed on Apr. 30, 2009.

(51) Int. Cl. C07H 17/02; C07H 15/04; C07H 15/26; C07H 15/203

(52) U.S. Cl. 536/17.4; 536/18.2; 536/17.8; 536/18.1

(57) ABSTRACT

Disclosed herein are processes for preparing glucopyranosyloxypyrazole derivatives. In particular, the present invention relates to glucopyranosyloxypyrazole derivatives having SGLT2 inhibitory activity and processes and intermediates for preparing the same.

The present invention relates to processes for the metal-catalyzed chemoselective preparation of esters and carbonates of pyranosyl derivatives. In particular, the present invention relates to glucopyranosyloxypyrazole derivatives having SGLT2 inhibitory activity and processes and intermediates for preparing the same.

### BACKGROUND OF THE INVENTION

Sodium dependent glucose transporters (SGLT), including SGLT1 and SGLT2, are membrane proteins that transports glucose. SGLT2 is mainly active in the proximal tubules of the kidney wherein it effects the transport of glucose from the urine into the bloodstream. The reabsorbed glucose is then utilized throughout the body. Diabetic patients are typically characterized by abnormal blood glucose levels. Consequently, inhibition of SGLT2 activity and therefore inhibition of glucose reabsorption in the kidneys is believed to be a possible mechanism for controlling blood glucose levels in such diabetic patients. Glucopyranosyloxypyrazole derivatives have been proposed for treatment of diabetic patients, with some being currently in clinical development. See U.S. Pat. Nos. 6,972,283; 7,084,123; 7,393,838; 6,815,428; 7,015,201; 7,247,616; and 7,256,209. Accordingly, scalable and cost efficient synthesis of glucopyranosyloxypyrazole derivatives as well as intermediates for producing the same is a current need in the pharmaceutical industry.

### BRIEF SUMMARY OF THE INVENTION

The present inventors have now discovered a highly chemoselective metal-catalyzed process for the esterification and alkoxycarbonylation of pyranosyl derivatives.

In one aspect of the present invention, there is provided a process for preparing a compound of formula (III),

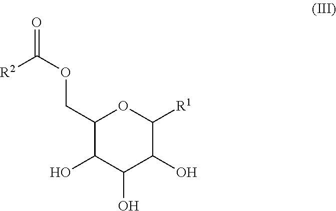

wherein:
R¹ is -Q-Q¹, wherein

Q is arylene, —O-arylene, heteroarylene, or O-heteroarylene, where each Q may be optionally substituted with one or more of C₁-C₆ alkyl or halo; and

Q¹ is aryl, alkaryl, or heteroaryl, wherein each Q¹ is optionally substituted with one or more of C₁-C₆ alkyl, C₁-C₆ haloalkyl, C₂-C₆ alkenyl, C₂-C₆ alkynyl, C₁-C₆ acyl, C₁-C₆ alkoxy, C₁-C₆ haloalkoxy, C₁-C₆ alkylthio, C₁-C₆ haloalkylthio, C₁-C₆ alkylamino, C₃-C₇ cycloalkyl, C₃-C₇ cycloalkyloxy, or halo; or

R¹ is C₁-C₆ alkoxy, aryl optionally substituted with —C₁-C₆ alkyl, —NO₂, or C(O)H, or —O-aryl optionally substituted with —C₁-C₆ alkyl, —NO₂, or C(O)H;
R² is —C₁-C₆ alkyl, C₁-C₆ alkoxy, —C₁-C₆ haloalkyl, —C₂-C₆ alkenyl, —C₂-C₆ alkynyl, aryl, alkaryl or heteroaryl;
comprising acylating or carbonating a pyranosyl derivative (IIIa):

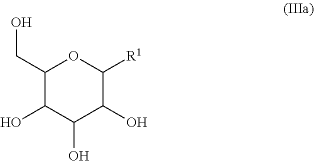

with a compound of formula (IV):

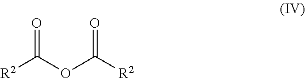

in the presence of a metal catalyst selected from a scandium or copper metal catalyst to provide a compound of formula (III).

In a second aspect of the present invention, there is provided a process for preparing a compound of formula (III),

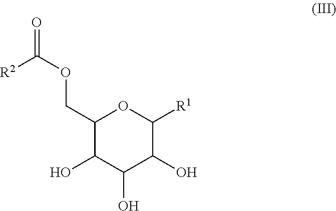

wherein:

### R¹ is

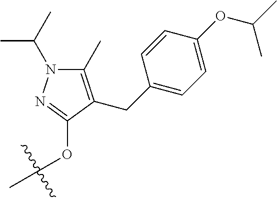

R² is ethoxy;
comprising:
(i) O-sulfonating a compound of formula (Ia)

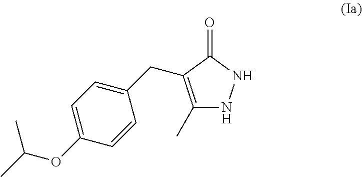

to produce a compound of formula (Ib);

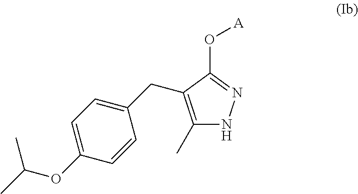

wherein A is a tosyl or mesyl group;
(ii) alkylating the compound of formula (Ib) to produce a compound of formula (Ic); and

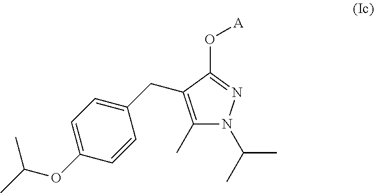

(iii) desulfonating the alkylated compound of formula (Ic) to produce a compound of formula (II);

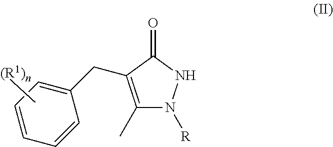

(iv) reacting a compound of formula (II) with a glucose derivative to provide a pyranosyl derivative of formula (IIIa); and

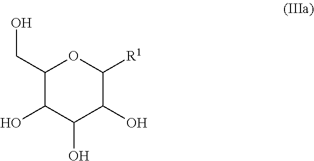

(v) acylating or carbonating the pyranosyl derivative of formula (IIIa): with a compound of formula (IV):

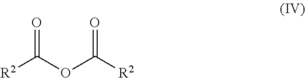

in the presence of a Sc or Cu catalyst to provide the compound of formula (III).

### DETAILED DESCRIPTION OF THE INVENTION

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “alkyl” refers to a straight or branched chain hydrocarbon, e.g., from one to twelve carbon atoms. Examples of “alkyl”, as used herein include, but are not limited to, methyl, ethyl, n-propyl, isopropyl, n-butyl, n-pentyl, and isobutyl, and the like.

As used herein, the term “C₁-C₆ alkyl” refers to an alkyl group, as defined above, which contains at least 1, and at most 6, carbon atoms. Examples of “C₁-C₆ alkyl” groups useful in the present invention include, but are not limited to, methyl, ethyl, n-propyl, isopropyl, isobutyl and n-butyl.

As used herein, the term “alkylene” refers to a straight or branched chain divalent hydrocarbon radical having from one to ten carbon atoms. Examples of “alkylene” as used herein include, but are not limited to, methylene, ethylene, n-propylene, n-butylene, and the like.

As used herein, the term “C₁-C₃ alkylene” refers to an alkylene group, as defined above, which contains at least 1, and at most 3, carbon atoms respectively. Examples of “C₁-C₃ alkylene” groups useful in the present invention include, but are not limited to, methylene, ethylene, n-propylene, isopropylene, and the like.

As used herein, the term “alkenyl” refers to a hydrocarbon group, e.g., from two to ten carbons, and having at least one carbon-carbon double bond. Examples of “alkenyl”, as used herein include, vinyl(ethenyl), propenyl, 2-methyl-1-propenyl, 1-butenyl, 2-butenyl, and isobutenyl.

As used herein, the term “C₂-C₆ alkenyl” refers to an alkenyl group, as defined above, containing at least 2, and at most 6, carbon atoms. Examples of “C₂-C₆ alkenyl” groups useful in the present invention include, but are not limited to, vinyl (ethenyl), propenyl, 2-methyl-1-propenyl, 1-butenyl, 2-butenyl, and isobutenyl.

As used herein, the term “alkynyl” refers to a hydrocarbon group, e.g., from two to ten carbons, and having at least one carbon-carbon triple bond. Examples of “alkynyl”, as used herein, include but are not limited to ethynyl(acetylenyl), 1-propynyl, 1-butynyl, 2-butynyl, 1-pentynyl, and 1-hexynyl.

As used herein, the term “C₂-C₆ alkynyl” refers to an alkynyl group, as defined above, containing at least 2, and at most 6, carbon atoms. Examples of “C₂-C₆ alkynyl” groups useful in the present invention include, but are not limited to, ethynyl (acetylenyl), 1-propynyl, 1-butynyl, 2-butynyl, 1-pentynyl, and 1-hexynyl.

As used herein, the terms “halo” refer to fluoro (—F), chloro (—Cl), bromo (—Br), or iodo (—I).

As used herein, the term “C₁-C₆ haloalkyl” refers to an alkyl group, as defined above, containing at least 1, and at most 6, carbon atoms substituted with at least one halo group, halo being as defined herein. Examples of “C₁-C₆ haloalkyl” groups useful in the present invention include, but are not limited to, methyl, ethyl, n-propyl, isopropyl, isobutyl and n-butyl substituted independently with one or more halo groups, e.g., fluoro, chloro, bromo and iodo.

As used herein, the term “alkoxy” refers to the group RₐO—, where Rₐ is alkyl as defined above and the term “C₁-C₆ alkoxy” refers to the group RₐO—, where Rₐ is C₁-C₆ alkyl as defined above. Examples of “C₁-C₆ alkoxy” groups useful in the present invention include, but are not limited to, methoxy, ethoxy, propyloxy, and isopropyloxy.

As used herein the term “C₁-C₆ haloalkoxy” refers to the group RₐO—, where Rₐ is C₁-C₆ haloalkyl as defined above. An exemplary C₁-C₆ haloalkoxy group useful in the present invention includes, but is not limited to, trifluoromethoxy.

As used herein, the term “alkylthio” refers to the group RₐS—, where Rₐ is alkyl as defined above and the term “C₁-C₆ alkylhio” refers to the group RₐS—, where Rₐ is C₁-C₆ alkyl as defined above. Examples of “C₁-C₆ alkylthio” groups useful in the present invention include, but are not limited to, methylthio, ethylthio, and propylthio.

As used herein, the term “C₁-C₆ haloalkylhio” refers to the group RₐS—, where Rₐ is C₁-C₆ haloalkyl as defined above. Examples of “C₁-C₆ haloalkylthio” groups useful in the present invention include, but are not limited to, methylthio, ethylthio, and propylthio wherein the alkyl is substituted independently with one or more halo groups, e.g., fluoro, chloro, bromo and iodo.

As used herein the term “C₁-C₆ alkylamino” refers to the group —NRₐR_{b} wherein Rₐ is —H or C₁-C₆ alkyl and R_{b} is —H or C₁-C₆ alkyl, where at least one of Rₐ and R_{b} is C₁-C₆ alkyl and C₁-C₆ alkyl is as defined above. Examples of “C₁-C₆ alkylamino” groups useful in the present invention include, but are not limited to, methylamino, ethylamino, propylamino, dimethylamino, and diethylamino.

As used herein, the term “C₃-C₇ cycloalkyl” refers to a non-aromatic hydrocarbon ring having from three to seven carbon atoms, which may or may not include a C₁-C₄ alkylene linker, through which it is attached, said linker being attached directly to the ring. Exemplary “C₃-C₇ cycloalkyl” groups include, but are not limited to, cyclopropyl, cyclobutyl, cyclopentyl, cyclohexyl, cycloheptyl, and cyclopropylmethylene.

As used herein, the term “C₃-C₇ cycloalkyloxy” refers to the group RₐO—, where Rₐ is C₃-C₇ cycloalkyl as defined above. Examples of “C₃-C₇ cycloalkyloxy” groups useful in the present invention include, but are not limited to, cyclopropyloxy, cyclobutyloxy, and cyclopentyloxy.

As used herein, the term “aryl” refers to a benzene ring or to a benzene ring system fused to one or more benzene or heterocyclyl rings to form, for example, anthracene, phenanthrene, napthalene, or benzodioxin ring systems. Examples of “aryl” groups include, but are not limited to, phenyl, 2-naphthyl, 1-naphthyl, biphenyl, 1,4-benzodioxin-6-yl as well as substituted derivatives thereof.

As used herein the term “−O-aryl” refers to an aryl group as defined above with an oxygen atom (O) linker group through which the aryl group may be attached.

As used herein, the term “arylene” refers to a benzene ring diradical or to a benzene ring system diradical wherein the benzene ring is fused to one or more benzene or heterocyclyl rings to form anthracenyl, napthalenyl, or benzodioxinyl diradical ring systems. Examples of “arylene” include, but are not limited to, benzene-1,4-diyl, naphthalene-1,8-diyl, anthracene-1,4-diyl, and the like.

As used herein the term “−O-arylene” refers to an arylene group as defined above with an oxygen atom (O) linker group through which the arylene group may be attached.

As used herein, the term “heteroaryl” refers to a monocyclic five to seven membered aromatic ring, or to a fused bicyclic or tricyclic aromatic ring system comprising two of such monocyclic five to seven membered aromatic rings. These heteroaryl rings contain one or more nitrogen, sulfur, and/or oxygen heteroatoms, where N-oxides and sulfur oxides and dioxides are permissible heteroatom substitutions. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinazolinyl, quinolinyl, isoquinolinyl, benzofuranyl, benzimidazolyl, benzothiophenyl, indolyl, indazolyl, and substituted versions thereof.

As used herein the term “−O-heteroaryl” refers to an heteroaryl group as defined above with an oxygen atom (O) linker group through which the heteroaryl group may be attached.

As used herein, the term “heteroarylene” refers to a five- to seven-membered aromatic ring diradical, or to a polycyclic heterocyclic aromatic ring diradical, containing one or more nitrogen, oxygen, or sulfur heteroatoms, where N-oxides and sulfur monoxides and sulfur dioxides are permissible heteroaromatic substitutions. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4-thiadiazole-2,5-diyl, 1,3-thiazole-2,4-diyl, 1,3-thiazole-2,5-diyl, pyrazole-3,4-diyl, pyridine-2,4-diyl, pyridine-2,3-diyl, pyridine-2,5-diyl, pyrimidine-2,4-diyl, quinoline-2,3-diyl, and the like.

As used herein the term “−O-heteroarylene” refers to an heteroarylene group as defined above with an oxygen atom (O) linker group through which the heteroarylene group may be attached.

As used herein, the term “aralkyl” refers to an aryl or heteroaryl group, as defined herein, attached through a C₁-C₃ alkylene linker, wherein the C₁-C₃ alkylene is as defined herein. Examples of “aralkyl” include, but are not limited to, benzyl, phenylpropyl, 2-pyridylmethyl, 3-isoxazolylmethyl, 5-methyl-3-isoxazolylmethyl, and 2-imidazolyl ethyl.

As used herein, the term “acyl” refers to the group RₐC(O)—, where Rₐ is alkyl as defined herein and the term “C₁-C₆ acyl” refers to the group RₐC(O)—, where Rₐ is C₁-C₆ alkyl as defined herein. Examples of “C₁-C₆ acyl” groups useful in the present invention include, but are not limited to, acetyl and propionyl.

As used herein, the term “alkoxycarbonyl” refers to the group RₐC(O)—, where Rₐ is alkoxy as defined herein and the term “C₁-C₆ alkoxycarbonyl” refers to the group RₐC(O)—, where Rₐ is C₁-C₆ alkoxy as defined herein. Examples of “C₁-C₆ alkoxycarbonyl” groups useful in the present invention include, but are not limited to, ethoxycarbonyl, methoxycarbonyl, n-propoxycarbonyl and isopropoxycarbonyl.

The present invention includes a process for preparing a compound of formula (III)

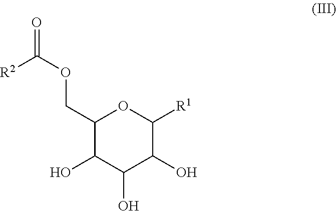

In one embodiment, R¹ is -Q-Q¹.

In one embodiment Q is arylene optionally substituted with one or more of C₁-C₆ alkyl or halo. In one embodiment Q is arylene optionally substituted with one or more halo. In one embodiment Q is phenylene optionally substituted with halo.

In one embodiment Q is —O-arylene optionally substituted with one or more of C₁-C₆ alkyl or halo. In one embodiment Q is —O-arylene. In one embodiment Q is —O-phenylene.

In one embodiment Q is heteroarylene optionally substituted with one or more of C₁-C₆ alkyl or halo. In one embodiment Q is heteroarylene optionally substituted with one or more C₁-C₆ alkyl. In one embodiment Q is pyrazole-diyl optionally substituted with one or more C₁-C₆ alkyl.

In one embodiment Q is O-heteroarylene optionally substituted with one or more of C₁-C₆ alkyl or halo. In one embodiment Q is heteroarylene optionally substituted with one or more C₁-C₆ alkyl. In one embodiment Q is pyrazole-diyl optionally substituted with one or more C₁-C₆ alkyl.

In one embodiment, Q¹ is aryl optionally substituted with one or more of C₁-C₆ alkyl, C₁-C₆ haloalkyl, C₂-C₆ alkenyl, C₂-C₆ alkynyl, C₁-C₆ acyl, C₁-C₆ alkoxy, C₁-C₆ haloalkoxy, C₁-C₆ alkylthio, C₁-C₆ haloalkylthio, C₁-C₆ alkylamino, C₃-C₇ cycloalkyl, C₃-C₇ cycloalkyloxy, or halo. In another embodiment, Q¹ is aryl optionally substituted with one or more C₁-C₆ alkyl, C₁-C₆ alkoxy, C₁-C₆ alkylthio, C₁-C₆ haloalkyl, or halo.

In another embodiment, Q¹ is aryl optionally substituted with one or more C₁-C₆ alkyl, C₁-C₆ alkoxy, or halo. In another embodiment, Q¹ is phenyl optionally substituted with one or more C₁-C₆ alkyl, C₁-C₆ alkoxy, or halo.

In one embodiment, Q¹ is aralkyl optionally substituted with one or more of C₁-C₆ alkyl, C₁-C₆ haloalkyl, C₂-C₆ alkenyl, C₂-C₆ alkynyl, C₁-C₆ acyl, C₁-C₆ alkoxy, C₁-C₆ haloalkoxy, C₁-C₆ alkylthio, C₁-C₆ haloalkylthio, C₁-C₆ alkylamino, C₃-C₇ cycloalkyl, C₃-C₇ cycloalkyloxy, or halo. In another embodiment, Q¹ is aralkyl optionally substituted with one or more C₁-C₆ alkyl, C₁-C₆ alkoxy, C₁-C₆ alkylthio, C₁-C₆ haloalkyl, or halo. In another embodiment, Q¹ is aralkyl optionally substituted with one or more C₁-C₆ alkyl, C₁-C₆ alkoxy, or halo. In another embodiment, Q¹ is benzyl optionally substituted with one or more C₁-C₆ alkyl, C₁-C₆ alkoxy, or halo.

In one embodiment, Q¹ is heteroaryl optionally substituted with one or more of C₁-C₆ alkyl, C₁-C₆ haloalkyl, C₂-C₆ alkenyl, C₂-C₆ alkynyl, C₁-C₆ acyl, C₁-C₆ alkoxy, C₁-C₆ haloalkoxy, C₁-C₆ alkylthio, C₁-C₆ haloalkylthio, C₁-C₆ alkylamino, C₃-C₇ cycloalkyl, C₃-C₇ cycloalkyloxy, or halo. In another embodiment, Q¹ is heteroaryl optionally substituted with one or more C₁-C₆ alkyl, C₁-C₆ alkoxy, C₁-C₆ alkylthio, C₁-C₆ haloalkyl, or halo. In another embodiment, Q¹ is aralkyl optionally substituted with one or more C₁-C₆ alkyl, C₁-C₆ alkoxy, or halo.

In another embodiment, R¹ is C₁-C₆ alkoxy; aryl optionally substituted with —C₁-C₆ alkyl, —NO₂, or C(O)H; or O-aryl optionally substituted with —C₁-C₆ alkyl, —NO₂, or C(O)H. In another embodiment, R¹ is C₁-C₆ alkoxy or O-aryl optionally substituted with —NO₂, or C(O)H. In another embodiment, R¹ is methoxy, phenoxy, p-nitrophenoxy, or phenoxy substituted with a formyl at the ortho position.

In one embodiment, R¹ is the substituent of formula (V):

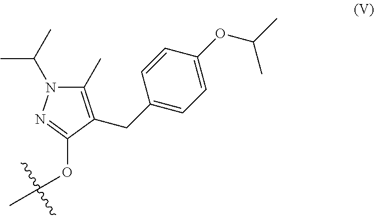

R¹ and compounds of formulae (Ia), (Ib), (Ic), (II), and (IIIa) may be prepared according to methods similar to those recited in Schemes 1-4.

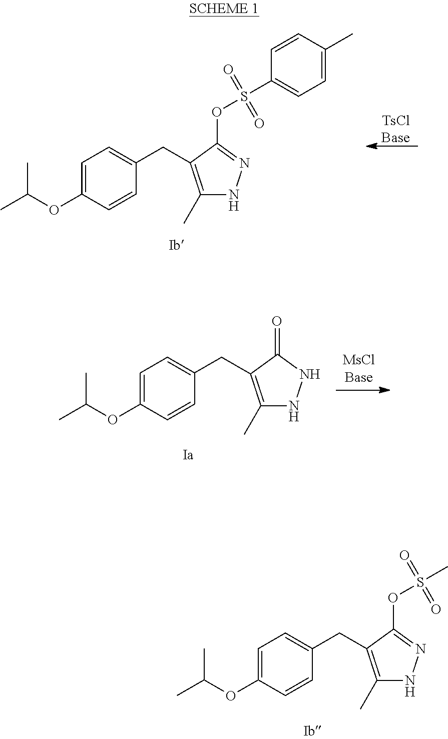

Scheme 1 illustrates the tosylation and mesylation of a compound of formula (Ia), wherein R¹ is the substituent of formula (V) above, to give sulfonated compounds of formula Ib′ and Ib″. These sulfonated compounds are the tosylated and mesylated forms of the specific compounds of formula (Ia) respectively. Tosylation of the compound of formula (Ia) was performed by reaction with tosyl chloride optionally in the presence of a base in a suitable solvent. The typical temperature range utilized was 15-30° C. Suitable solvents include, but are not limited to, N,N-dimethylformamide (DMF), acetonitrile (MeCN), dichloromethane (CH₂Cl₂), and ethyl acetate (EtOAc). Bases which may be utilized include, but are not limited to, cesium carbonate (Cs₂CO₃), potassium carbonate (K₂CO₃), pyridine, and triethylamine (Et₃N). Mesylation of the compound of formula (Ia) was performed by reaction with methanesulfonyl chloride or methanesulfonic anhydride optionally in the presence of a base in a suitable solvent. Suitable solvents include, but are not limited to, N,N-dimethylformamide, (DMF), acetonitrile (MeCN), and n-methyl pyrrolidinone (NMP). Bases which may be utilized include, but are not limited to, pyridine, triethylamine (Et₃N), and lithium hydroxide (LiOH). Isolatable solids are obtainable for both tosyl and mesyl intermediates. Mono-sulfonation is obtained by using no added base or a very weak base such as pyridine. Accordingly, in one embodiment, the tosylation or mesylation takes place in the presence of a weak base, for instance pyridine. In another embodiment, the tosylation or mesylation takes place without use of a base. The O-sulfonated intermediates of formula (Ib′) and (Ib″) alkylate on nitrogen with good regioselectivity. Typically regioselectivity of about 10:1 is observed.

The O-sulfonated compound of formula (Ib), for example the compound of formula (Ib′) or (Ib″), is then alkylated to form a compound of formula I(c) and then the compound of formula I(c) is deprotected (desulfonated) to form a compound of formula (II). In this instance R¹ is again the substituent of formula (V). Scheme 2 depicts the alkylation (isopropylation) and deprotection of the compound of formula (Ib′), i.e., the tosyl protected intermediate.

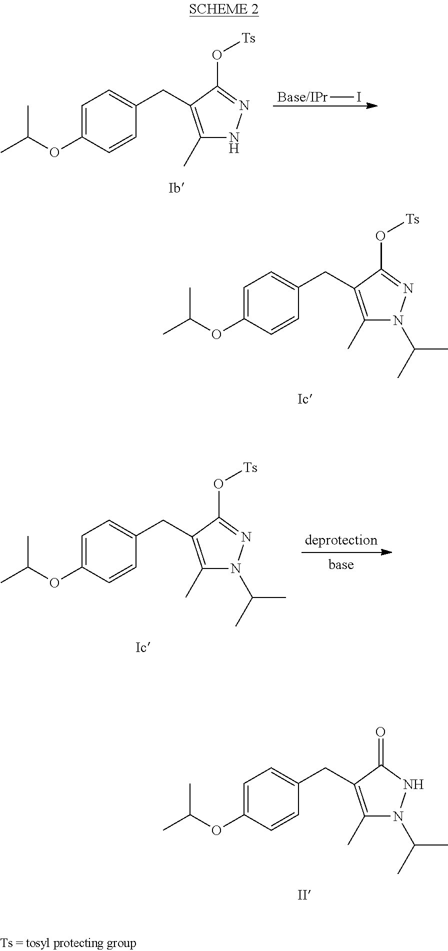

Alkylation of the compound of formula (Ib′) proceeds with reaction with an alkyl halide, for instance isopropyl iodide, in the presence of a base in a suitable solvent. The alkylation reaction is typically run at 20-30° C. Bases which may be utilized include, but are not limited to, potassium carbonate (K₂CO₃), 1,8-diazabicyclo[5.4.0]undec-7-ene (DBU), potassium tert-butoxide (KOtBu), triethylamine (Et₃N), lithium hydroxide (LiOH), cesium carbonate (Cs₂CO₃), sodium tert-butoxide (NaOtBu), potassium hydroxide (KOH), and pyridine). Suitable solvents include N,N-dimethylformamide (DMF), acetonitrile (MeCN), dichloromethane (CH₂Cl₂). Ratios achieved are on the order of 10:1 regioselectivity. Decomposition of excess alkyl halide via reaction with ethanolamine or other nucleophile may be performed prior to deprotection of O-sulfonate. Deprotection (desulfonation) proceeds by reaction with a base, such as NaOH, at a temperature of about 60-70° C. to arrive at the compound of formula II′.

Scheme 3 depicts alkylation and deprotection of the compound of formula (Ib″), i.e., the mesyl protected intermediate.

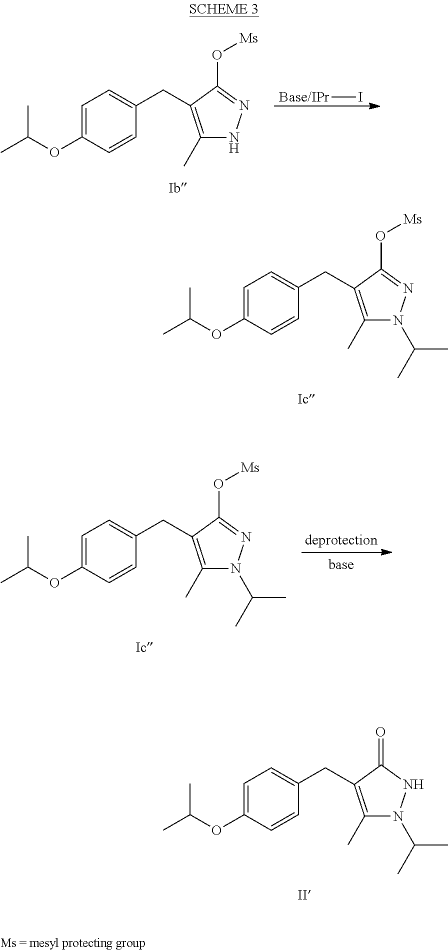

Alkylation of the compound of formula (Ib″) proceeds with reaction with an alkyl halide, for instance isopropyl iodide, in the presence of a base in a suitable solvent. The alkylation reaction is typically run at 20-30° C. Usable bases include, but are not limited to, lithium hydroxide (LiOH), sodium hydroxide (NaOH), potassium hydroxide (KOH), potassium tert-butoxide (KOtBu), cesium carbonate (Cs₂CO₃), potassium carbonate (K₂CO₃), sodium tert-butoxide (NaOtBu), lithium tert-butoxide (LiOtBu), lithium carbonate (Li₂CO₃), and sodium carbonate (Na₂CO₃). Suitable solvents include, but are not limited to, N,N-dimethylformamide (DMF), N-methylpyrrolidinone (NMP), N,N-dimethylacetamide (DMAC) and acetonitrile (MeCN). Prior to deprotection, decomposition of excess alkyl halide via reaction with ethanolamine or other nucleophile may be performed prior to deprotection of O-sulfonate. Deprotection (desulfonation) proceeds by reaction with a base, such as NaOH, at a temperature of about 60-70° C. to arrive at the compound of formula II″.

Typical alkylating agents which may be utilized to effect the alkylation of the starting compounds of Schemes 2 or 3 are alkyl halides. Specific alkylating agents for isopropylation of the starting compounds of Schemes 2 and 3, including isopropyl halides, may be as follows:

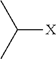

where X is —Cl, —F, —Br, —I, or —OR⁶ where R⁶ is mesyl, tosyl, or nosyl.

In one embodiment, the alkylating agent is isopropyl iodide.

In one embodiment, the alkylation reaction is quenched with a mild base, for example, ethanolamine to destroy the remaining isopropyl iodide prior to deprotection in order to protect against bis-alkylation.

Typical mild bases which may be utilized to quench the alkylation reaction to avoid bis-alkylation, include compounds of the following structures:

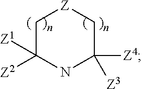

wherein:
Z¹, Z², Z³, and Z⁴ are independently H, C₁-C₆ alkyl, C₃-C₇ cycloalkyl, or aryl,

### Z is CH₂, N, O, or S, and

n is 0 to 3;

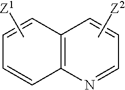

wherein:
Z¹ and Z² are independently selected from —H, C₁-C₆ alkyl, aryl, C₃-C₇ cycloalkyl, —F, —Cl, and —Br;

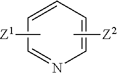

Z¹ and Z² are independently selected from —H, C₁-C₆ alkyl, aryl, C₃-C₇ cycloalkyl, —F, —Cl, and —Br;

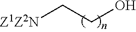

Z¹ and Z² are independently selected from —H, C₁-C₆ alkyl, C₃-C₇ cycloalkyl, and aryl,
n is 0 to 3;

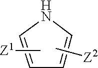

Z¹ and Z² are independently selected from —H, C₁-C₆ alkyl, aryl, C₃-C₇ cycloalkyl, —F, —Cl, or —Br;

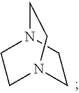

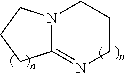

n is 0 to 3;
and
Z¹Z²Z³N wherein Z¹, Z², Z³ are independently selected from —H, C₁-C₆ alkyl, C₃-C₇ cycloalkyl, or aryl.

Once prepared, the compound of formula (II) may be glyclosidated to form a pyranosyl derivative of formula (IIIa).

Scheme 4 depicts one embodiment of such a glucosidation.

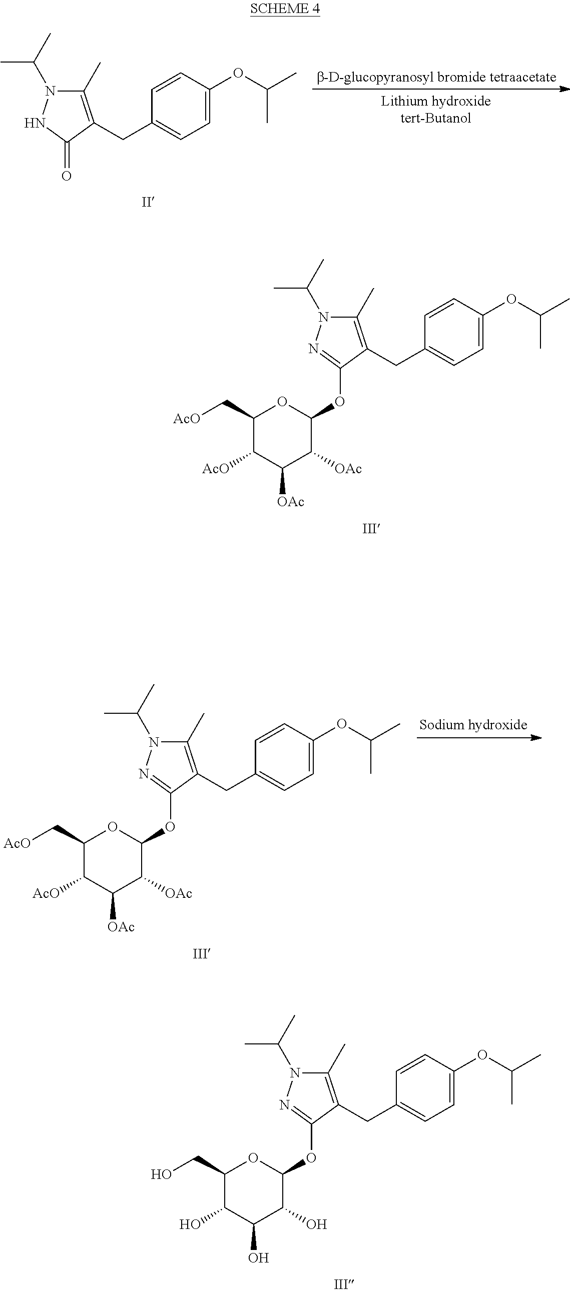

The glucosidation or glycosylation of the compound of formula II, in this embodiment a compound of Formula II′, is typically carried out using a protected and anomerically activated glucose derivative in the presence of a base in a suitable solvent to form a compound of Formula III′. The compound of formula III′ is then hydrolyzed with a strong base, such as sodium hydroxide, to cleave the acetyl protecting groups to arrive at the compound of formula III″. Both reactions are carried out at a temperature of about 35 to 40° C. Protecting groups which may be utilized include, but are not limited to, acetyl and pivaloyl. Activating groups which may be utilized include, but are not limited to chloride and bromide. Inorganic bases which may be utilized include, but are not limited to, sodium hydride, lithium hydroxide, sodium hydroxide, potassium hydroxide, cesium hydroxide, lithium carbonate, sodium carbonate, potassium carbonate, and cesium carbonate. Organic bases which may be utilized include, but are not limited to lithium tert-butoxide, sodium tert-butoxide, potassium tert-butoxide, tert-butyl lithium, lithium diisopropyl amide, and lithium hexamethyldisilazane. Suitable solvents which may be utilized include, but are not limited to toluene, acetone, 2-butanone, methyl-isobutyl ketone, ethanol, methanol, isopropanol, butanol, tert-butanol, neopentanol, tetrahydrofuran, 2-methyl tetrahydrofuran, methyl tert-butyl ether, and dichloromethane. The glycosidation is very selective for the O-position of compound II.

In another embodiment, R¹ is

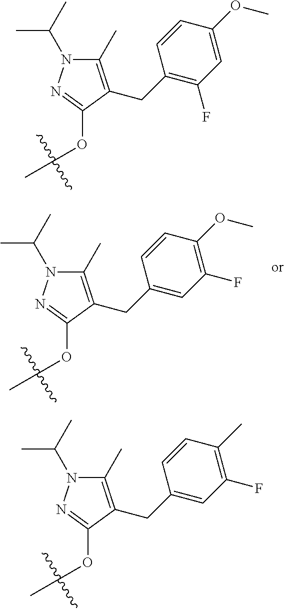

These R¹ substituents and compounds containing the same may be prepared according to procedures similar to those disclosed in U.S. Pat. No. 6,815,428.

In another embodiment, R¹ is

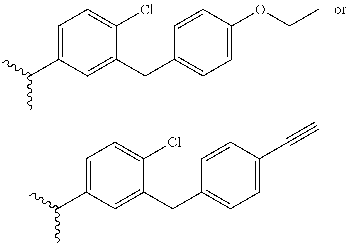

These R¹ substituents and compounds containing the same may be prepared according to procedures similar to those disclosed in U.S. Pat. No. 6,515,117 or WO 05/092877.

R¹ may be attached to the anomeric carbon of the pyranose derivative of formula (III) such that the α or β anomers result. In one embodiment, R¹ is attached in a manner such that the α anomer results. In another embodiment, R¹ is attached in a manner such that the β anomer results.

The pyranose derivative of formula (III) may be in the D or L configuration and each of the substituents attached at C₁-C₅ may be of the (R) or (S) configuration. Specific examples of pyranose derivatives of formula (III) include:

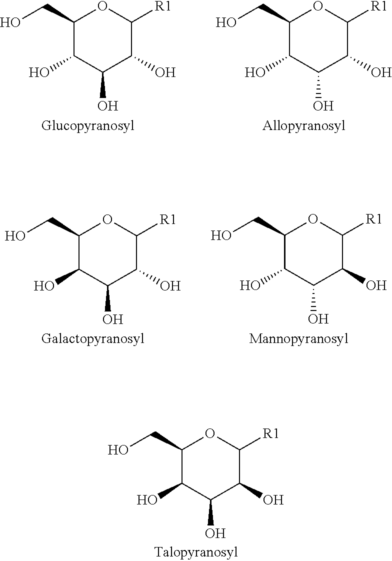

The compound of formula (IIIa) is then acylated or carbonated

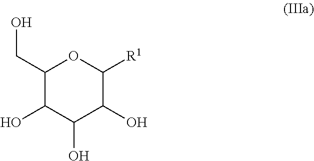

with a compound of formula (IV):

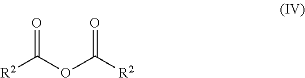

In one embodiment, R² is —C₁-C₆ alkyl, C₁-C₆ alkoxy, —C₁-C₆ haloalkyl, —C₂-C₆ alkenyl, —C₂-C₆ alkynyl, aryl, alkaryl or heteroaryl. In another embodiment, R² is —C₁-C₆ alkyl, —C₁-C₆ alkoxy, or aryl. In one embodiment, R² is —C₁-C₆ alkoxy. In one embodiment, R² is -methyl, ethoxy, methoxy, 1,1-dimethylethyloxy, or phenyl. In another embodiment, R² is ethoxy.

As recited above the process of the present invention is carried out in the presence of a metal catalyst which is a scandium or a copper metal catalyst. Suitable catalyst include but are not limited to Sc(OTf)₃, ScCl₃, ScBr₃, CuOTf, Cu(OTf)₂, CuBr, CuBr₂, Cu(BF₄)₂, The reaction is typically run at 20-70° C. Suitable solvents include, but are not limited to, toluene, ethanol, methanol, 2-propanol, t-butanol, tetrahydrofuran, 2-methyltetrahydrofuran, Methyl-tert-butyl ether (MTBE), acetone, and methyl isobutyl ketone.

In one embodiment, the metal catalyst is a scandium metal catalyst. In another embodiment, the metal catalyst is copper metal catalyst. In one embodiment, the metal catalyst is Sc(OTf)₃. Scheme 5 illustrates one embodiment of such a carbonation reaction.

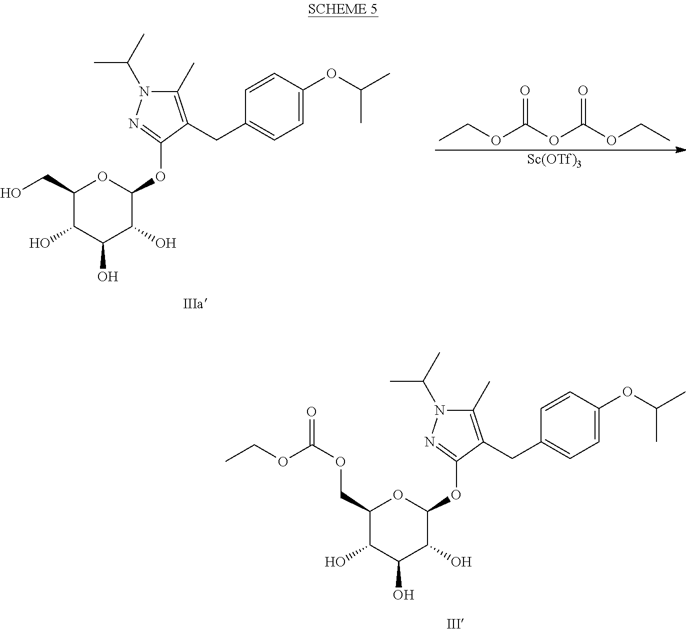

Certain embodiments of the present invention will now be illustrated by way of example only. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
g (grams);
mg (milligrams);
L (liters);
mL (milliliters);
μL (microliters);
psi (pounds per square inch);
M (molar);
mM (millimolar);
N (normal);
Hz (Hertz);
Vol (volumes)
MHz (megahertz);
mol (moles);
mmol (millimoles);
RT (room temperature);
RP (reverse phase);
min (minutes);
h (hours);
mp (melting point);
TLC (thin layer chromatography);
Tᵣ (retention time);
MeOH (methanol);
PrOH (isopropanol);
HOAc (acetic acid);
TEA (triethylamine);
TFA (trifluoroacetic acid);
THF (tetrahydrofuran);
NMP (n-methylpyrrolidinone)
DMSO (dimethylsulfoxide);
EtOAc (ethyl acetate);
DME (1,2-dimethoxyethane);
DCM (dichloromethane);
DCE (dichloroethane);
DMF (N,N-dimethylformamide);
atm (atmosphere);
HPLC (high pressure liquid chromatography);

Unless otherwise indicated, all temperatures are expressed in ° C. (degrees Centigrade). All reactions conducted under an inert atmosphere at room temperature unless otherwise noted.

¹H NMR spectra were recorded on a Varian VXR-300, a Varian Unity-300, a Varian Unity-400 instrument, a Varian VNMRS-500, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), h (heptet), q (quartet), m (multiplet), br (broad).

Low-resolution mass spectra (MS) were recorded on a JOEL JMS-AX505HA, JOEL SX-102, Agilent series 1100MSD, or a SCIEX-APliii spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60E-254), visualized with UV light, 5% ethanolic phosphomolybdic acid or p-anisaldehyde solution. Flash column chromatography was performed on silica gel (230-400 mesh, Merck). Optical rotations were obtained using a Perkin Elmer Model 241 Polarimeter. Melting points were determined using a MeI-Temp II apparatus and are uncorrected.

### Example 1

### Preparation of 5-methyl-1-(1-methylethyl)-4-({-4-[(1-methylethyl)oxy]phenyl}methyl)-1H-pyrazol-3-yl 6-O-[(ethyloxy)carbonyl]-β-D-glucopyranoside (2)

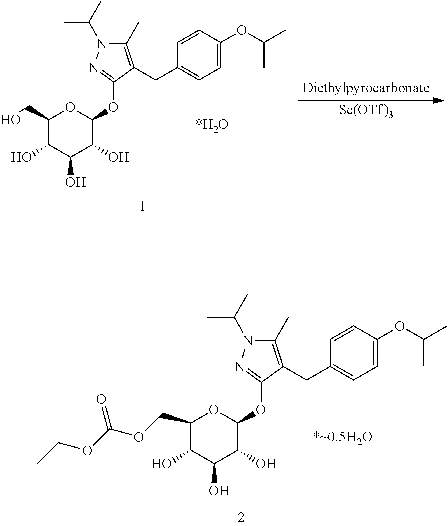

To a solution of 1 (1 Kg, 1.0 eq, 2.1 mol) in toluene (6.4 L) and IMS (1.6 L) is added scandium triflate (1.6 g, 0.0015 eq) and diethylpyrocarbonate (398 g, 1.15 eq). The solution is heated to 45-55° C. for 1-6 hours before quenching with dilute acetic acid (3.0 L, 2.5 vol %). The mixture is cooled to 20° C. and the layers are allowed to separate. The bottom layer (aqueous) is discarded. The organic layer is washed again with dilute aq. acetic acid (3 L) and the aqueous layer discarded. The final organic layer is then concentrated under reduced pressure to about 2.25 volumes. MIBK (2.75 L), water (31 mL), and heptanes (8.5 L) are added and the desired compound is isolated by crystallization to afford a white solid. The cake is washed with 25% MIBK in heptanes and then dried under reduced pressure (30° C.) to afford the title compound 2 as a white solid (1.03 kg, 92% yield). ¹H NMR (DMSO-d₆, 500 MHz, 25° C.) 1.17 (t, J=7.1 Hz, 3H), 1.22 (d, J=6.1 Hz, 6H), 1.27 (dd, J₁=6.7 Hz, J₂=8.3 Hz, 6H), 2.06 (s, 3H), 3.12-3.29 (m, 3H), 3.38 (ddd, J₁=1.8 Hz, J₂=6.1 Hz, J₃=10.0 Hz, 1H), 3.51 (s, 2H), 4.08 (q, J=7.1 Hz, 2H), 4.10 (dd, J₁=6.1 Hz, J₂=11.7 Hz, 1H), 4.29 (dd, J₁=1.8 Hz, J₂=11.7 Hz, 1H), 4.34 (sp, J=6.4 Hz, 1H), 4.50 (sp, J=6.0 Hz, 1H), 5.12 (d, J=7.9 Hz, 1H), 5.14 (d, J=5.3 Hz, 1H), 5.25 (d, J=5.8 Hz, 1H), 5.32 (d, J=5.4 Hz, 1H), 6.75 (d, J=8.6 Hz, 2H), 7.08 (d, J=8.6 Hz, 2H). ¹³C NMR (DMSO-d₆, 125 MHz, 25° C.) 9.1, 13.9, 21.8, 21.9, 22.2, 26.2, 48.3, 63.4, 66.6, 68.9, 69.5, 73.2, 73.8, 76.3, 100.6, 102.8, 115.3, 129.0, 133.2, 135.5, 154.4, 155.3, 157.8.

### Example 2

### Preparation of 5-methyl-1-(1-methylethyl)-4-({-4-[(1-methylethyl)oxy]phenyl}methyl)-1H-pyrazol-3-yl 6-O-{[(1,1-dimethylethyl)oxy]carbonyl}-β-D-glucopyranoside (3)

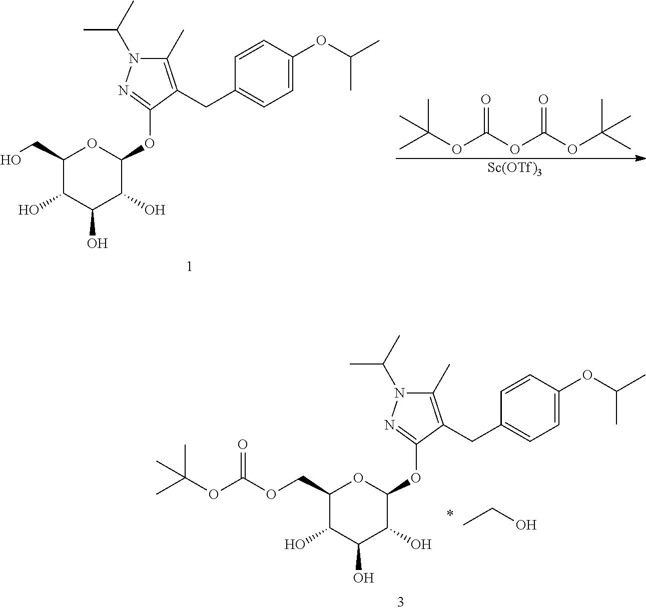

To a solution of 1 (5 g, 1.0 eq, 10.7 mmol) in acetone (25 mL) is added scandium triflate (53 mg, 0.01 eq) and di-tert-butyl dicarbonate (2.68 g, 1.15 eq). The solution is heated to 45-55° C. for 6 hours. The solvent is removed via vacuum distillation to an oily residue. Ethanol (50 mL) and heptane (25 mL) are charged and the batch is heated to 5° C. Solids form during the heat up. The batch is cooled to 25 C and filtered. The solids are washed with ethanol:heptane 2:1 (25 mL) and dried in a vacuum oven at 30° C. to afford the title compound 3 as a white solid (5.43 g, 92% yield). ¹H NMR (500 MHz, DMSO-d₆) δ ppm 1.22 (d, J=6.05 Hz, 6H) 1.28 (dd, J=11.68, 6.54 Hz, 6H) 1.38 (s, 9H) 2.06 (s, 3H) 3.14 (td, J=9.11, 5.75 Hz, 1H) 3.20 (ddd, J=8.80, 5.38 Hz, 1H) 3.25 (dt, J=8.86, 5.14 Hz, 1H) 3.34-3.40 (m, 1H) 3.51 (s, 2H) 4.01 (dd, J=11.62, 6.05 Hz, 1H) 4.23 (dd, J=11.34, 1.25 Hz, 1H) 4.28-4.39 (m, 1H) 4.50 (m, J=12.04, 6.02, 6.02, 6.02, 6.02 Hz, 1H) 5.11 (d, J=5.01 Hz, 1H) 5.14 (d, J=7.76 Hz, 1H) 5.21 (d, J=5.69 Hz, 1H) 5.29 (d, J=5.20 Hz, 1H) 6.75 (d, J=8.56 Hz, 2H) 7.08 (d, J=8.50 Hz, 2H), Ethanol δ ppm 1.06 (t, J=7.00 Hz, 3H) 3.45 (qd, J=6.95, 5.20 Hz, 2H) ¹³C NMR (126 MHz, CDCl₃) δ ppm 9.07, 21.78, 21.79, 22.00, 22.25, 26.23, 27.23, 48.28, 65.92, 68.86, 69.53, 73.21, 73.87, 76.33, 81.22, 100.52, 102.76, 115.25, 128.99, 133.14, 135.53, 152.84, 155.29, 157.73, Ethanol δ ppm 18.46, 55.94.

### Example 3

### Preparation of 5-methyl-1-(1-methylethyl)-4-({-4-[(1-methylethyl)oxy]phenyl}methyl)-1H-pyrazol-3-yl 6-O-acetyl-β-D-glucopyranoside (4)

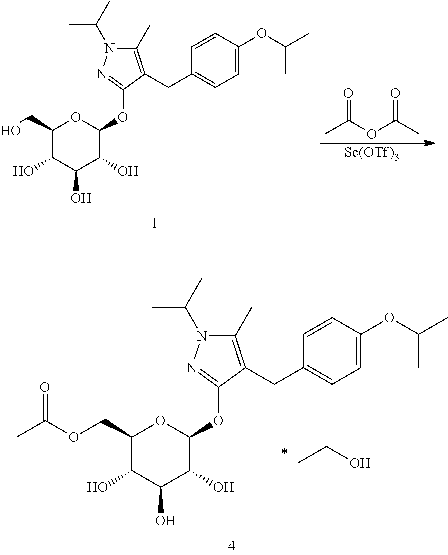

To a solution of 1 (5 g, 1.0 eq, 10.7 mmol) in 2-methyl-tetrahydrofuran (40 mL) is added scandium triflate (53 mg, 0.01 eq) and acetic anhydride (1.42 g, 1.3 eq). The solution is heated to 45-55° C. for 2 hours. The solvent is removed via vacuum distillation to an oily residue. Ethanol (30 mL) and heptane (50 mL) are charged and the batch is heated to 5° C. Solids form upon cooling. The batch is cooled to 25 C and filtered. The solids are washed with 10% ethanol/heptane (40 mL) and dried in a vacuum oven at 30° C. to afford the title compound 4 as a white solid (3.7 g, 68% yield). ¹H NMR (500 MHz, DMSO-d₆) δ ppm 1.22 (d, J=6.05 Hz, 6H) 1.28 (t, J=5.99 Hz, 6H) 1.95 (s, 3H) 2.06 (s, 3H) 3.15 (td, J=8.96, 5.81 Hz, 1H) 3.17-3.29 (m, 2H) 3.37 (td, J=9.11, 1.22 Hz, 1H) 3.52 (s, 2H) 4.02 (dd, J=11.83, 6.51 Hz, 1H) 4.24 (dd, J=11.80, 1.34 Hz, 1H) 4.34 (m, J=12.99, 6.49, 6.49, 6.49, 6.49 Hz, 1H) 4.50 (m, J=12.04, 5.99, 5.99, 5.99, 5.99 Hz, 1H) 5.05-5.15 (m, 2H) 5.19 (d, J=5.62 Hz, 1H) 5.28 (d, J=5.07 Hz, 1H) 6.75 (m, J=8.56 Hz, 2H) 7.07 (m, J=8.50 Hz, 2H) Ethanol δ ppm 1.06 (t, J=7.00 Hz, 3H) 3.45 (qd, J=6.95, 5.20 Hz, 2H) ¹³C NMR (126 MHz, DMSO-d₆) δ ppm 9.06, 20.51, 21.78, 21.78, 22.01, 22.18, 26.17, 48.22, 63.53, 68.88, 69.80, 73.21, 73.88, 76.35, 100.54, 102.66, 115.24, 115.24, 128.96, 128.96, 133.17, 135.52, 155.28, 157.77, 170.13

### Example 4

### Preparation of 5-methyl-1-(1-methylethyl)-4-({4-[(1-methylethyl)oxy]phenyl}methyl)-1H-pyrazol-3-yl 6-O-(phenylcarbonyl)-β-D-glucopyranoside (5)

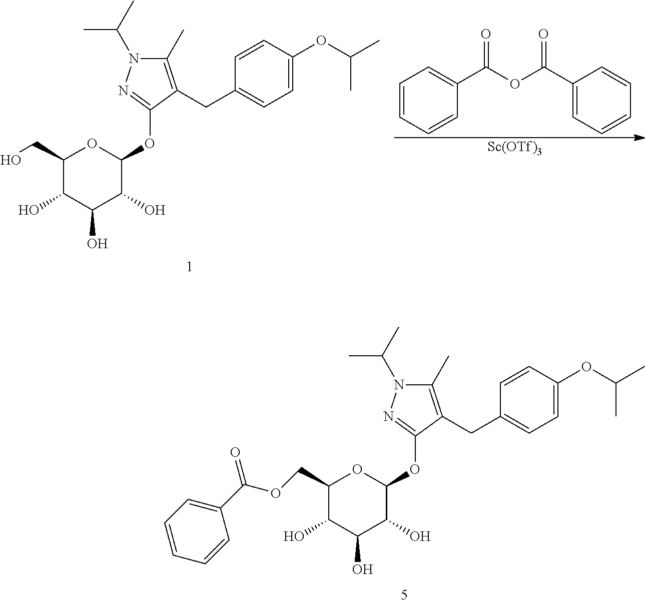

To a solution of 1 (5 g, 1.0 eq, 10.7 mmol) in 2-methyl-tetrahydrofuran (25 mL) is added scandium triflate (53 mg, 0.01 eq) and benzoic anhydride (3.62 g, 1.5 eq). The solution is heated to 45-55° C. for 24 hours. The solvent is removed via vacuum distillation to an oily residue. Ethanol (50 mL) is charged and the batch is heated to 5° C. Solids form upon addition of ethanol at room temperature. The batch is cooled to 25 C and filtered. The solids are washed with ethanol (15 mL). Ethanol (50 mL) is charged and the batch is heated to 5° C. The solids are washed with ethanol (15 mL) and dried in a vacuum oven at 30° C. to afford the title compound 5 as a white solid (4.6 g, 75% yield). ¹H NMR (500 MHz, DMSO-d₆) δ ppm 1.14-1.22 (m, 9H) 1.25 (d, J=6.48 Hz, 3H) 2.02 (s, 3H) 3.24-3.37 (m, 3H) 3.51 (s, 2H) 3.55 (t, J=7.83 Hz, 1 H) 4.23-4.35 (m, 2H) 4.40 (spt, J=6.02 Hz, 1H) 4.56 (d, J=11.62 Hz, 1H) 5.20 (d, J=7.70 Hz, 1H) 5.32 (br. s., 3H) 6.67 (m, J=8.44 Hz, 2H) 7.06 (m, J=8.19 Hz, 2H) 7.48 (t, J=7.79 Hz, 2H) 7.64 (t, J=7.46 Hz, 1H) 7.92 (d, J=8.01 Hz, 2H) ¹³C NMR (126 MHz, DMSO-d₆) δ ppm 9.02, 21.72, 21.75, 21.86, 22.11, 26.19, 48.20, 64.26, 68.83, 69.96, 73.25, 73.99, 76.37, 100.79, 102.91, 115.15, 115.15, 128.48, 128.92, 128.92, 129.00, 129.60, 133.11, 133.17, 135.42, 155.22, 157.81, 165.53.

### Example 5

### Preparation 6-O-[(ethyloxy)carbonyl]-αD-phenylglucopyranoside (7)

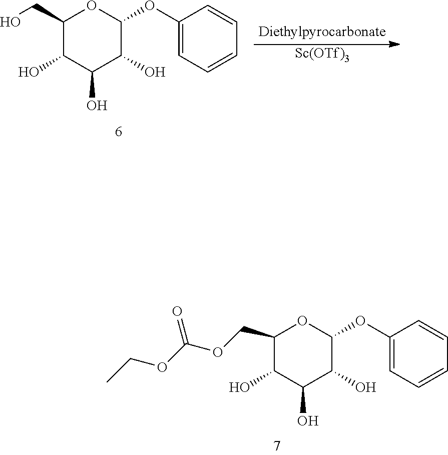

To a solution of 6 (150 mg, 1.0 eq, 0.57 mmol) in toluene (1.1 mL) and ethanol (0.4 mL) is added scandium triflate (3 mg, 0.1 eq) and diethylpyrocarbonate (104 mg, 1.1 eq). The solution is heated to 45-55° C. for 1-6 hours before concentrating to dryness. The residue was chromatographed on silica (1:1 heptane:ethyl acetate) to afford the title compound 7 as a white solid (168 mg, 87% yield).

¹H NMR (DMSO-d₆, 500 MHz, 25° C.) 1.17 (t, J=7.1, 3H), 3.16-3.22 (m, 1H), 3.38-3.43 (m, 1H), 3.61-3.67 (m, 1H), 3.72 (ddd, J₁=2.0 Hz, J₂=6.2 Hz, J₃=10.0 Hz, 1H), 3.97-4.10 (m, 2H), 4.14 (dd, J₁=6.1 Hz, J₂=11.6 Hz, 1H), 4.26 (dd, J₁=2.1 Hz, J₂=11.7 Hz, 1H), 5.05 (d, J=5.2 Hz, 1H), 5.13 (d, J=6.3 Hz, 1H), 5.29 (d, J=5.9 Hz, 1H), 5.40 (d, J=3.7 Hz, 1H), 7.01 (t, J=7.3 Hz, 1H), 7.06 (d, J=8.2 Hz, 2H), 7.30 (t, J=7.8 Hz, 2H). ¹³C NMR (DMSO-d₆, 125 MHz, 25° C.) 14.0, 63.4, 66.6, 69.8, 70.4, 71.3, 72.8, 97.6, 116.8, 121.9, 129.3, 154.3, 156.9.

### Example 6

### 6-O-[(ethyloxy)carbonyl]-βD-methylglucopyranoside (9)

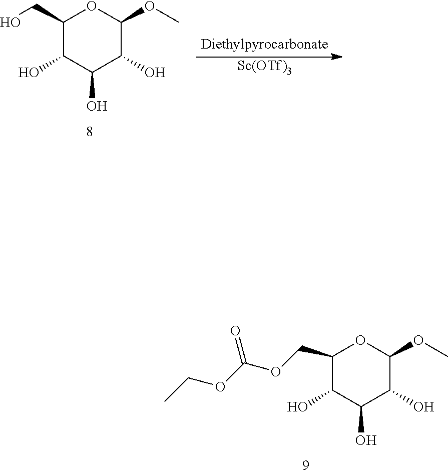

The title compound was prepared by heating a heterogeneous mixture of Methyl-β-D-glucopyranose 8 (10 g, 51.5 mmol), Ethanol (100 mL, 10 volumes), Scandium triflate (253 mg, 0.51 mmol), and diethylpyrocarbonate (8.35 g, 51.5 mmol) to 50° C. The reaction mixture was held for two hours during which time the solids dissolved completely into a colorless solution and significant off-gassing was observed. The solution was cooled and the solvent removed via vacuum distillation to give a quantitative yield of greater than 95% purity of a single product as a colorless oil that solidified to a white solid upon standing, 9. For characterization purposes, the material was chromatographed over silica using 5% Methanol/Dichloromethane as a diluent. ¹H NMR (500 MHz, DMSO-d₆) δ ppm 1.21 (t, J=7.09 Hz, 3H) 3.02-3.10 (m, J=9.78, 8.93, 5.81 Hz, 1H) 3.17-3.23 (m, J=9.84, 6.36, 3.67 Hz, 1H) 3.25 (s, 3H) 3.34-3.41 (m, J=9.17, 9.17, 4.95 Hz, 1H) 3.50-3.56 (m, J=9.78, 6.11, 1.65 Hz, 1H) 4.11 (t, J=7.21 Hz, 2H) 4.13 (dd, J=6.17, 3.97 Hz, 1H) 4.31 (dd, J=11.43, 1.83 Hz, 1H) 4.54 (d, J=3.61 Hz, 1H) 4.78 (d, J=6.42 Hz, 1H) 4.86 (d, J=5.01 Hz, 1H) 5.16 (d, J=5.81 Hz, 1H); ¹³C NMR (125 MHz, DMSO-d₆) δ ppm: 14.01, 54.33, 63.43, 66.88, 69.29, 69.99, 71.67, 73.04, 99.65, 154.46.

### Example 7

### Preparation 6-O-acetoxy-β-D-phenylglucopyranoside (10)

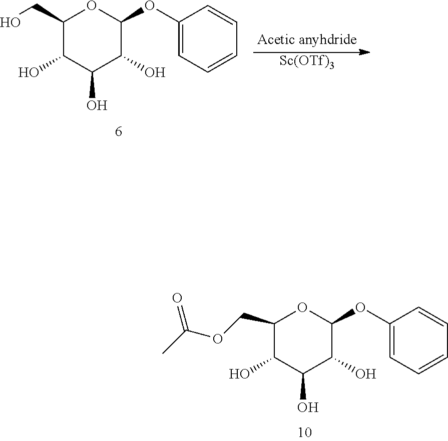

The title compound was prepared by heating a heterogeneous mixture of phenyl-D-glucopyranose 6 (1 g, 3.6 mmol), 2-methyltetrahydrofuran (100 mL, 100 volumes) and ethanol (10 mL, 10 volumes) to 50° C. at which point the solids dissolved. Scandium triflate (19 mg, 0.04 mmol), and acetic anhydride (0.74 g, 7.3 mmol) were charged and the reaction was held at 50° C. for 2 hours. The solution was cooled and solids crystallized out of solution. The solids were filtered, washed with ethanol and dried under vacuum. The filtrate was concentrated to an oil weighing 0.6 g that showed 85% product by NMR. The crystalline solids 10 were analyzed by NMR. ¹H NMR (300 MHz, DMSO-d₆) δ ppm 2.00 (s, 3H) 3.09-3.32 (m, 3H) 3.60 (ddd, J=9.46, 7.02, 2.08 Hz, 1H) 4.07 (dd, J=11.84, 6.71 Hz, 1H) 4.27 (dd, J=11.84, 2.08 Hz, 1H) 4.90 (d, J=7.57 Hz, 1H) 5.20 (d, J=4.64 Hz, 1H) 5.29 (d, J=5.37 Hz, 1H) 5.39 (d, J=4.88 Hz, 1H) 6.93-7.05 (m, 3H) 7.23-7.36 (m, 2H).

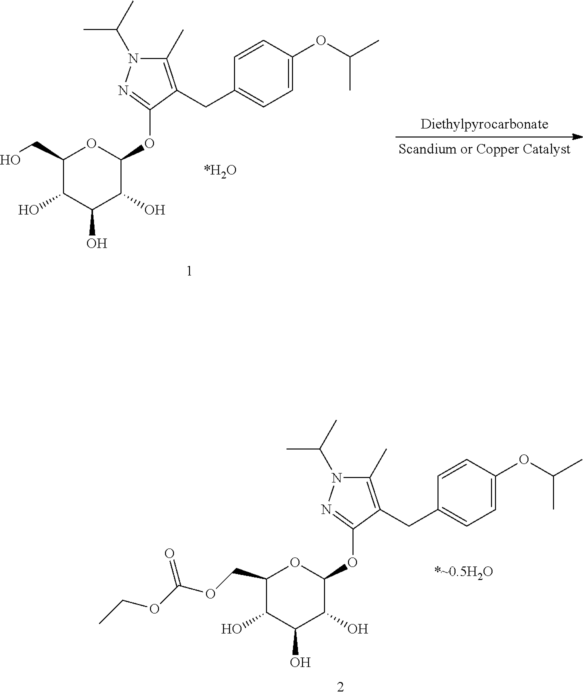

### Example 8

### Preparation of 5-methyl-1-(1-methylethyl)-4-({-4-[(1-methylethyl)oxy]phenyl}methyl)-1H-pyrazol-3-yl 6-O-{[(1,1-dimethylethyl)oxy]carbonyl}-β-D-glucopyranoside (3)

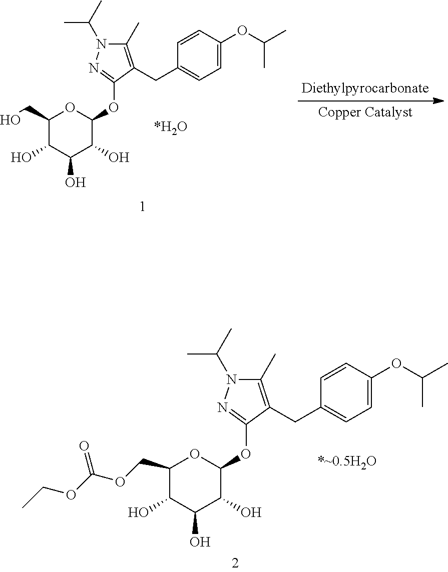

Copper (II) triflate catalyst: To a solution of 1 (15.6 g, 1.0 eq, 34.6 mmol) in t-butanol (80 ml) is added copper II triflate (0.125 g, 0.01 eq) and diethylpyrocarbonate (6.2 g, 1.1 eq). The solution is heated to 45-55° C. for 1-7 hours before concentration to dryness. The residue is diluted with toluene and washed with water. The toluene solution is crystallized as above to afford the title compound 2 as a white solid (85% yield).

¹H NMR (DMSO-d₆, 500 MHz, 25° C.) 1.17 (t, J=7.1 Hz, 3H), 1.22 (d, J=6.1 Hz, 6H), 1.27 (dd, J₁=6.7 Hz, J₂=8.3 Hz, 6H), 2.06 (s, 3H), 3.12-3.29 (m, 3H), 3.38 (ddd, J₁=1.8 Hz, J₂=6.1 Hz, J₃=10.0 Hz, 1H), 3.51 (s, 2H), 4.08 (q, J=7.1 Hz, 2H), 4.10 (dd, J₁=6.1 Hz, J₂=11.7 Hz, 1H), 4.29 (dd, J₁=1.8 Hz, J₂=11.7 Hz, 1H), 4.34 (sp, J=6.4 Hz, 1H), 4.50 (sp, J=6.0 Hz, 1H), 5.12 (d, J=7.9 Hz, 1H), 5.14 (d, J=5.3 Hz, 1H), 5.25 (d, J=5.8 Hz, 1H), 5.32 (d, J=5.4 Hz, 1H), 6.75 (d, J=8.6 Hz, 2H), 7.08 (d, J=8.6 Hz, 2H). ¹³C NMR (DMSO-d₆, 125 MHz, 25° C.) 9.1, 13.9, 21.8, 21.9, 22.2, 26.2, 48.3, 63.4,

TABLE 1
following summarized yields and selectivity for some of the
compounds prepared in Examples 1-8 as well as for additional compounds prepared
according to methods similar to those of Examples 1-8..

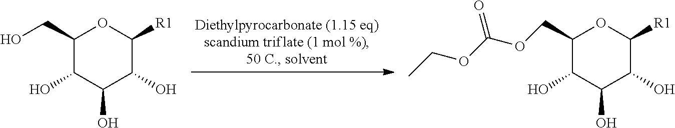

| | | | | Isolated |
|---|---|---|---|---|
| Product | Solvent | Time | Selectivity¹ | Yield |
| 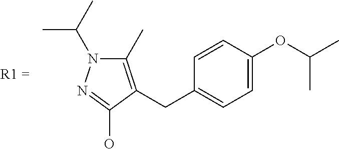 | toluene: ethanol 4:1 | 2 hr | >97% | 93%² |
| R1 = OPh | toluene: | 1 hr | 95% | 85% |
| | ethanol 3:1 | | | |
| R1 = OMe | ethanol | 1 hr | 95% | 95% |
| 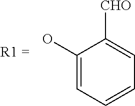 | toluene: ethanol 4:1 | 1 hr | 92% | 78% |
| 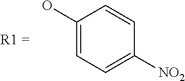 | toluene: ethanol 4:1 | 1 hr | 94% | 74% |
| 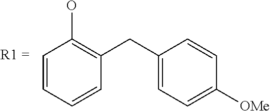 | toluene: ethanol 4:1 | 2 hr | 95% | 81% |
| 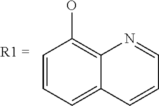 | ethanol | 6 hr | ? | 74% |
| 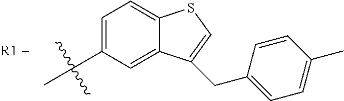 | toluene: ethanol 5:1 | 2 hr | 95% | 78% |
| 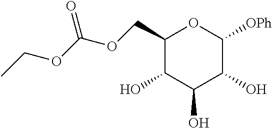 | toluene: ethanol 4:1 | 1 hr | 95% | 87% |
| 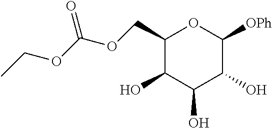 | toluene: ethanol 2:1 | 3 hr³ | 94% | 63% |

¹Selectivity = 100 x (desired monocarbonate/desired product + starting material + byproducts).
²Used 0.15 mol % scandium triflate.
³Temperature = 40 C.

We claim:

1. A process for preparing a compound of formula (III), 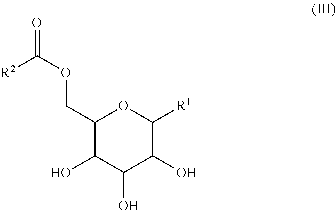
wherein:
R¹ is -Q-Q¹, wherein
Q is arylene, —O-arylene, heteroarylene, or O-heteroarylene, where each Q may be optionally substituted with one or more of C₁-C₆ alkyl or halo; and
Q¹ is aryl, alkaryl, or heteroaryl, wherein each Q¹ is optionally substituted with one or more of C₁-C₆ alkyl, C₁-C₆ haloalkyl, C₂-C₆ alkenyl, C₂-C₆ alkynyl, C₁-C₆ acyl, C₁-C₆ alkoxy, C₁-C₆ haloalkoxy, C₁-C₆ alkylthio, C₁-C₆ haloalkylthio, C₁-C₆ alkylamino, C₃-C₇ cycloalkyl, C₃-C₇ cycloalkyloxy, or halo; or
R¹ is C₁-C₆ alkoxy, aryl optionally substituted with —C₁-C₆ alkyl, —NO₂, or C(O)H, or -D-aryl optionally substituted with —C₁-C₆ alkyl, —NO₂, or C(O)H;
R² is —C₁-C₆ alkyl, C₁-C₆ alkoxy, —C₁-C₆ haloalkyl, —C₂-C₆ alkenyl, —C₂-C₆ alkynyl, aryl, alkaryl or heteroaryl;
comprising acylating or carbonating a pyranosyl derivative (IIIa): 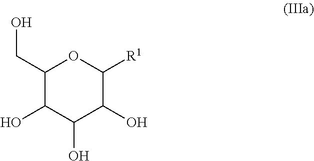
with a compound of formula (IV): 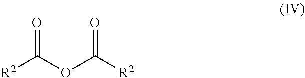
in the presence of a metal catalyst selected from a scandium or copper metal catalyst to provide a compound of formula (III).

2. A process for preparing a compound of formula (III),
wherein: 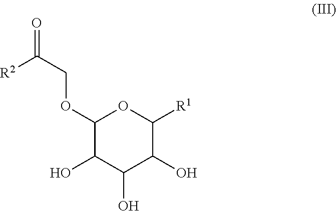
R¹ is 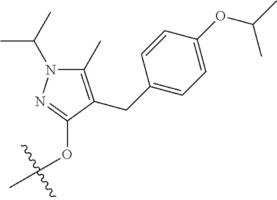
R² is ethoxy;
comprising:
(i) O-sulfonating a compound of formula (Ia) 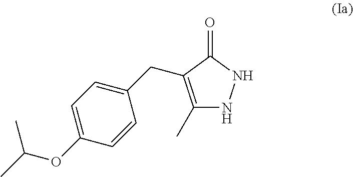
to produce a compound of formula (Ib); 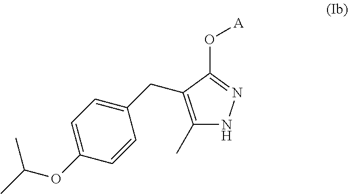
wherein A is a tosyl or mesyl group;
(iv) alkylating the compound of formula (Ib) to produce a compound of formula (Ic); and 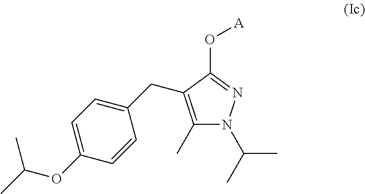
(v) desulfonating the alkylated compound of formula (Ic) to produce a compound of formula (II); 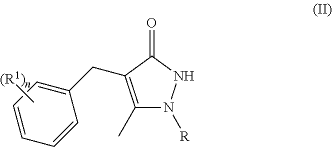
(iv) reacting a compound of formula (II) with a glucose derivative to provide a pyranosyl derivative of formula (IIIa); and 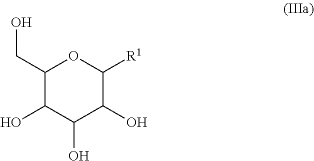
(v) acylating or carbonating the pyranosyl derivative of formula (IIIa):
with a compound of formula (IV): 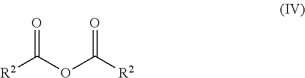
in the presence of a Sc or Cu catalyst to provide the compound of formula (III).
